(19) United States
(12) Patent Application Publication
(10) Pub. No.: US 2024/0407991 A1
(43) Pub. Date: Dec. 12, 2024

(54) LOW PENETRATION SUNSCREEN COMPOSITIONS

(71) Applicant: Johnson & Johnson Consumer Inc., Skillman, NJ (US)

(72) Inventors: Lívea Fujita Barbosa, São José dos Campos (BR); Susan Daly, Basking Ridge, NJ (US); Indaiá Lara Gehlen, São Paulo (BR); Ricardo Segovia-Borray, São José dos Campos (BR); Maria Carolina Semprini, São José dos Campos (BR); Cinthia Satake, São José dos Campos (BR); Camilla Floriano Mendes de Oliveira, São José dos Campos (BR); Rodrigo Collina Romanhole, Curitiba-Paraná (BR)

(21) Appl. No.: 18/653,546

(22) Filed: May 2, 2024

Related U.S. Application Data
(60) Provisional application No. 63/471,362, filed on Jun. 6, 2023.

(51) Int. Cl. A61K 8/49; A61K 8/29; A61K 8/41; A61K 8/67; A61K 8/73; A61Q 17/04

(52) U.S. Cl. CPC A61K 8/4966; A61K 8/29; A61K 8/411; A61K 8/496; A61K 8/675; A61K 8/735; A61Q 17/04

(57) ABSTRACT

The present invention provides a sunscreen composition comprising: (a) diethylamino hydroxybenzoyl hexyl benzoate; (b) bis-ethylhexyloxyphenol methoxyphenyl triazine; (c) ethylhexyl triazone; and (d) methylene bis-benzotriazolyl tetramethylbutylphenol; wherein the sunscreen composition is substantially free of UV absorbers having a molecular mass of 380 Daltons or less.

This application claims the benefit of U.S. provisional application 63/471,362 filed on Jun. 6, 2023, the complete disclosure of which is hereby incorporated herein by reference for all purposes.

### BACKGROUND OF THE INVENTION

It is well known that prolonged exposure to ultraviolet radiation, such as from the sun, can lead to the formation of light dermatoses and erythemas, and increase the risk of skin cancers, such as melanoma. Exposure to UV radiation also accelerates skin aging, such as loss of skin elasticity and wrinkling.

For these reasons, sunscreen compositions are commonly used to provide protection from the sun, and a variety of sunscreen compositions are commercially available. Many contain low molecular weight, organic UV absorbers such as octocrylene, homosalate, avobenzone, oxybenzone, octyl salicylate and cinnamates. While these UV filters are effective for absorbing UV radiation, high amounts are often required to provide a high SPF sunscreen composition. Because they are low molecular weight compounds, consumers are sometimes concerned about their potential to penetrate the skin, particularly when used in high amounts.

HIRUDO Derm Sun Protect Krem dlya Ditei Sontsezakhysnyi SPF 75 Solar Protect Kids contains a combination of UV absorbers with titanium dioxide, a UV blocker. The UV absorber combination is diethylamino hydroxybenzoyl hexyl benzoate, ethylhexyl triazone, methylene bis-benzotriazolyl tetramethylbutylphenol, tris-biphenyl triazine, and bis-ethylhexyloxyphenol methoxyphenyl triazine along with the low molecular weight UV absorber ethylhexyl methoxycinnamate.

LUPIN Propiacne Day Loção Hidratante com Proteção Solar SPF 35 also contains a combination of UV absorbers with titanium dioxide. The UV absorbers are diethylamino hydroxybenzoyl hexyl benzoate, tris-biphenyl triazine, bis-ethylhexyloxyphenol methoxyphenyl triazine, methylene BIS-benzotriazolyl tetramethylbutylphenol, and ethylhexyl triazone. However, the product also contains the low molecular weight UV absorber octocrylene.

Sunscreen compositions substantially free of low molecular weight UV absorbers, such as ethylhexyl methoxycinnamate and octocrylene, and therefore substantially non-penetrating to the skin, have now been discovered. Such compositions instead contain a specific combination of high molecular weight UV absorbers in certain, low amounts, but still desirably provide high SPF products.

### SUMMARY OF THE INVENTION

The present invention provides a sunscreen composition comprising: (a) diethylamino hydroxybenzoyl hexyl benzoate; (b) bis-ethylhexyloxyphenol methoxyphenyl triazine; (c) ethylhexyl triazone; and (d) methylene bis-benzotriazolyl tetramethylbutylphenol; wherein the sunscreen composition is substantially free of UV absorbers having a molecular mass of 380 Daltons or less.

The composition may optionally further comprise up to about 3 weight percent of tris-biphenyl triazine.

The composition may optionally further comprise up to about 2 weight percent of a UV blocker such as titanium dioxide or zinc oxide.

The composition may optionally further comprise a polymeric UV absorber, for example a linear UV-absorbing polyether comprising a covalently bound benzotriazole chromophores.

The composition may optionally further comprise one or more cosmetically acceptable active agents, such as niacinamide, glycerin, acetyl glucosamine, and sodium hyaluronate.

### DETAILED DESCRIPTION OF THE INVENTION

Unless defined otherwise, all technical and scientific terms used herein have the same meaning as commonly understood by one of ordinary skill in the art to which the invention belongs. All publications, patent applications, patents, and other references mentioned herein are incorporated by reference.

As used herein, “topically applying” means directly laying on or spreading on outer skin or the scalp, e.g., by use of the hands or an applicator such as a wipe, roller, or spray.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin or hair.

As used herein, “cosmetically effective amount” means an amount of a physiologically active compound or composition sufficient for treating one or more conditions, but low enough to avoid serious side effects. The cosmetically effective amount of the compound or composition will vary with the condition being treated, the age and physical condition of the end user, the severity of the condition being treated/prevented, the duration of the treatment, the nature of other treatments, the specific compound or product/composition employed, the cosmetically acceptable carrier utilized, and like factors.

As used herein, “cosmetically acceptable” means that the ingredients the term describes are suitable for use in contact with tissues (e.g., the skin or hair) without undue toxicity, incompatibility, instability, irritation, allergic response, or the like.

As used herein, a “cosmetically acceptable active agent” is a compound (synthetic or natural) that has a cosmetic or therapeutic effect on the skin or hair.

As used herein, “treatment or treating” refers to mitigating, reducing, preventing, improving, or eliminating the presence or signs of a condition or disorder.

As used herein, “phase-stable” means the water and oil phases of an emulsion do not appreciably separate at room temperature for at least two weeks.

As used herein, “substantially free of” means not intentionally or deliberately added. For example, a composition may be substantially free of an ingredient it if contains less than 1% by weight, or less than 0.5% by weight, or less than 0.1% by weight, of such ingredient. A composition may contain none of such ingredient, i.e., 0% by weight and be completely free of such ingredient.

As used herein, “UV absorber” means a compound that absorbs radiation in some portion of the ultraviolet spectrum (290 nm-400 nm), such as one having an extinction coefficient of at least about 1000 mol⁻¹ cm⁻¹, for at least one wavelength within the above-defined ultraviolet spectrum.

Unless otherwise indicated, a percentage or concentration refers to a percentage or concentration by weight (i.e., % (W/W). Unless stated otherwise, all ranges are inclusive of the endpoints, e.g., “from 4 to 9” includes the endpoints 4 and 9.

### UV Absorbers

The sunscreen composition of the invention comprises a combination of UV absorbers each having a molecular mass greater than 380 Daltons. In particular, the sunscreen composition comprises: (a) diethylamino hydroxybenzoyl hexyl benzoate; (b) bis-ethylhexyloxyphenol methoxyphenyl triazine; (c) ethylhexyl triazone; and (d) methylene bis-benzotriazolyl tetramethylbutylphenol.

The combination of UV absorbers in the sunscreen composition may consist essentially of, or consist of: (a) diethylamino hydroxybenzoyl hexyl benzoate; (b) bis-ethylhexyloxyphenol methoxyphenyl triazine; (c) ethylhexyl triazone; and (d) methylene bis-benzotriazolyl tetramethylbutylphenol.

The sunscreen composition of the invention may comprise UV absorbers, UV blockers and a cosmetically acceptable topical carrier, wherein the UV absorbers and UV blockers consist essentially of, or consist of: (a) about 2 to about 6 weight percent of diethylamino hydroxybenzoyl hexyl benzoate; (b) 3 weight percent or less of tris-biphenyl triazine; (c) about 0.5 to about 3.5 weight percent of bis-ethylhexyloxyphenol methoxyphenyl triazine; (d) about 1.64 to about 4.55 weight percent of ethylhexyl triazone; (e) about 2 to about 6 weight percent of methylene bis-benzotriazolyl tetramethylbutylphenol; and (f) 2 weight percent or less of titanium dioxide.

The sunscreen composition is substantially free of UV absorbers having a molecular mass of 380 Daltons and below. Preferably, the sunscreen composition is completely free of UV absorbers having a molecular mass of 380 Daltons and below.

Accordingly, the sunscreen composition comprises only UV absorbers having molecular masses greater than 380 Daltons. It may optionally also comprise one or more UV blockers or polymeric UV absorbers, as described below. However, it is substantially free of UV absorbers having a molecular mass of 380 Daltons or less. In this manner, the sunscreen composition is advantageously low penetrating or substantially non-penetrating to skin or hair.

Optionally, all the UV absorbers in the sunscreen composition have melting points above 50° C.

The total amount of UV absorbers in the sunscreen composition ranges from about 9 to about 20 weight percent, preferably about 9.5 to about 17.5 weight percent, of the total weight of the sunscreen composition. The total amount of UV absorbers in the sunscreen composition may be less than about 18 weight percent of the total weight of the sunscreen composition. More preferably, the total amount of UV absorbers in the sunscreen composition is less than about 15 weight percent of the total weight of the sunscreen composition.

Despite the low total amount of UV absorbers in the sunscreen composition, the composition nevertheless provides high SPF values.

### Diethylamino Hydroxybenzoyl Hexyl Benzoate (“DHHB”)

The first UV absorber in the sunscreen composition is diethylamino hydroxybenzoyl hexyl benzoate (“DHHB”). Diethylamino hydroxybenzoyl hexyl benzoate is also known chemically as benzoic acid, 2-[−4-(diethylamino)-2-hydroxybenzoyl]-, hexylester. It is commercially available, for example, as UVINUL® A-PLUS from BASF or PARSOL® DHHB from DSM. The molecular mass of diethylamino hydroxybenzoyl hexyl benzoate is 398 Daltons.

The sunscreen composition preferably contains about 2 to about 6 weight percent of diethylamino hydroxybenzoyl hexyl benzoate based on the total weight of the sunscreen composition.

More preferably the sunscreen composition contains about 3 to about 5.5 weight percent of diethylamino hydroxybenzoyl hexyl benzoate based on the total weight of the sunscreen composition.

### Bis-Ethylhexyloxyphenol Methoxyphenyl Triazine (“BEMT”)

The second UV absorber in the sunscreen composition is bis-ethylhexyloxyphenol methoxyphenyl triazine (“BEMT”). Bis-ethylhexyloxyphenol methoxyphenyl triazine is also known chemically as phenol, 2,2′-[6-(4-methoxyphenyl)-1,3,5-triazine-2,4-diyl]bis(5-((2-ethylhexyl)oxy; (1,3,5)-triazine-2,4-bis{[4-(2-ethyl-hexyloxy)-2-hydroxy]-phenyl}-6-(4-methoxyphenyl); anisotriazine; and bemotrizinol. It is commercially available, for example, from BASF as Tinosorb® S. The molecular mass of bis-ethylhexyloxyphenol methoxyphenyl triazine is 627 Daltons.

The sunscreen composition preferably contains about 0.5 to about 3.5 weight percent of bis-ethylhexyloxyphenol methoxyphenyl triazine based on the total weight of the sunscreen composition.

More preferably the sunscreen composition contains about 1.5 to about 2.3 weight percent of bis-ethylhexyloxyphenol methoxyphenyl triazine based on the total weight of the sunscreen composition.

### Ethylhexyl Triazone (“EHT”)

The third UV absorber in the sunscreen composition is ethylhexyl triazone (“EHT”). Ethylhexyl triazone is also known chemically as 4-[[4,6-bis[[4-(2-ethylhexoxy-oxomethyl)phenyl]amino]-1,3,5-triazin-2-yl]amino]benzoic acid 2-ethylhexyl ester; and octyl triazone. It is commercially available, for example, from BASF as UVINUL® T 150. The molecular mass of ethylhexyl triazone is 823 Daltons.

The sunscreen composition preferably contains about 1.64 to about 4.55 weight percent of ethylhexyl triazone based on the total weight of the sunscreen composition.

### Methylene Bis-Benzotriazolyl Tetramethylbutylphenol (“MBBT”)

The fourth UV absorber in the sunscreen composition is methylene bis-benzotriazolyl tetramethylbutylphenol (“MBBT”). Methylene bis-benzotriazolyl tetramethylbutylphenol is also known chemically as phenol, 2,2′-methylenebis[6-(2H-benzotriazol-2-yl)-4-(1,1,3,3-tetramethylbutyl), and bisoctrizole. It is commercially available, for example, as Tinosorb® M from BASF. The molecular mass of methylene bis-benzotriazolyl tetramethylbutylphenol is 659 Daltons.

The sunscreen composition preferably contains about 2 to about 6 weight percent of methylene bis-benzotriazolyl tetramethylbutylphenol based on the total weight of the sunscreen composition.

More preferably the sunscreen composition contains about 2 to about 5.5 weight percent of methylene bis-benzotriazolyl tetramethylbutylphenol based on the total weight of the sunscreen composition.

### Tris-Biphenyl Triazine (“TBT”)

The composition may optionally contain the additional UV absorber tris-biphenyl triazine (“TBT”). Tris-biphenyl triazine is also known chemically as 1,3,5-Triazine, 2,4,6-tris([1,1′-biphenyl]-4-yl)-, and 2,4,6-tri([1,1′-biphenyl]-4-yl)-1,3,5-triazine. It is commercially available as Tinosorb® A2B from BASF. The molecular mass of tris-biphenyl triazine is 537 Daltons.

When used, the sunscreen composition preferably contains 3 weight percent or less of tris-biphenyl triazine based on the total weight of the sunscreen composition.

More preferably the sunscreen composition contains about 0.5 to about 1 weight percent of tris-biphenyl triazine based on the total weight of the sunscreen composition.

### Low Molecular Weight UV Absorbers

The sunscreen composition is substantially free, preferably completely free of UV absorbers having a molecular mass of 380 Daltons or less.

Examples of UV absorbers having a molecular mass of 380 Daltons or less include: octocrylene, homosalate, avobenzone, octyl salicylate, methoxycinnamate derivatives such as octyl methoxycinnamate and isoamyl methoxycinnamate; camphor derivatives such as 4-methyl benzylidene camphor, camphor benzalkonium methosulfate, ecamsule (Mexoryl® SX); terephthalylidene dicamphor sulfonic acid, and terephthalylidene dicamphor sulfonic acid; other salicylate derivatives such as trolamine salicylate and ethylhexyl salicylate; sulfonic acid derivatives such as phenylbenzimidazole sulfonic acid; benzone derivatives such as dioxybenzone, sulisobenzone, and oxybenzone; benzoic acid derivatives such as aminobenzoic acid and octyldimethyl para-amino benzoic acid; octocrylene and other β,β-diphenylacrylates; dioctyl butamido triazone; octyl triazone; drometrizole trisiloxane; and menthyl anthranilate.

Optionally, the sunscreen composition is substantially free of UV absorbers having a melting point under 30° C.

The sunscreen composition may be free of octocrylene.

The sunscreen composition may be free of homosalate.

The sunscreen composition may be free of avobenzone.

The sunscreen composition may be free of octyl salicylate.

The sunscreen composition may be free of ethylhexyl methoxycinnamate.

The sunscreen composition may be free of phenylbenzimidazole sulfonic acid.

### UV Blocker

The sunscreen composition may optionally contain a UV blocker. As used herein, “UV blocker” means a compound that reflects or scatters UV radiation. UV blockers are typically inorganic metallic oxides, including titanium dioxide, zinc oxide, and other transition metal oxides. They are typically solid particles in a micronized or nanonized form having a diameter from about 0.01 micron to about 10 microns.

The sunscreen composition may contain 2 weight percent or less of a UV blocker based on the total weight of the sunscreen composition.

The UV blocker may be selected from the group consisting of titanium dioxide and zinc oxide.

The UV blocker may be titanium dioxide. An example of a titanium dioxide that may be used is Titanium Dioxide (and) Silica, commercially available from Merck as Eusolex® T AVO.

### Polymeric UV Absorber

The sunscreen composition may also optionally comprise a polymeric UV absorber.

The polymeric UV absorber may be a linear UV absorbing polyether, i.e., a polymer comprising a backbone of ether functional groups and UV absorbing chromophores covalently bound to the backbone.

As used herein, “linear UV absorbing polyether” means a polyether that absorbs radiation in some portion of the ultraviolet spectrum (wavelengths between 290 and 400 nm). The linear UV absorbing polyether has a weight average molecular weight (Mw), which may be suitable for reducing or preventing the chromophore from absorbing through the skin. According to one embodiment, a suitable molecular weight for the linear UV absorbing polyether is Mw greater than 500. In one embodiment, Mw is in the range of about 500 to about 50,000. In another embodiment, the Mw is in the range of about 1,000 to about 20,000, such as from about 1,000 to about 10,000.

Such linear UV absorbing polyethers are typically contained in polymer compositions composed of a complex combination of different molecules, i.e., a complex reaction product. Typically, these polymer compositions comprise at least about 50% or more of the linear UV-absorbing polyether. The polymer compositions may comprise at least about 75% of the linear UV-absorbing polyether. The polymer compositions may comprise at least about 90% of the linear UV-absorbing polyether, such as about 95% or more.

Linear UV absorbing polyethers include a plurality of ether functional groups covalently bonded to each other. The “backbone” of the linear UV absorbing polyether refers to the longest continuous sequence of covalently bonded ether functional groups. Other smaller groups of covalently bonded atoms are considered pendant groups that branch from the backbone.

The linear UV absorbing polyether preferably has a backbone that is unbranched.

The linear UV absorbing polyether may include glyceryl repeat units and accordingly, may be characterized as a polyglycerol. As used herein, “glyceryl repeat units” (also referred to herein “glyceryl remnant units”) means glycerol units excluding nucleophilic groups such as hydroxyl groups. Glyceryl remnant units include ether functional groups, and generally may be represented as C₃H₅O for linear and dendritic remnants (Rokicki et al. Green Chemistry., 2005, 7, 52). Suitable glyceryl remnant units include dehydrated forms (i.e. one mole of water removed) of the following glyceryl units: linear-1,4 (L1,4) glyceryl units; linear-1,3 (L1,3) glyceryl repeat units; dendritic (D) glyceryl units; terminal-1,2 (T1,2) units; and terminal-1,3 (T1,3) units.

According to certain embodiments, the linear UV-absorbing polyether includes either or both of the repeat units shown in FORMULA IA and FORMULA IB, below:

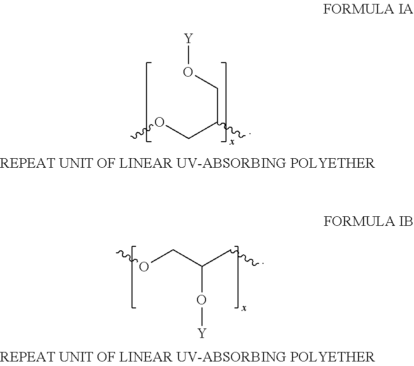

In FORMULAS IA and IB, X may be selected from, for example, hydrogen, linear alkyl, alkenyl or alkynyl hydrocarbon chains, linear siloxanes, and the like. In one embodiment the group X represents octadecane.

Y represents a UV-chromophore, described further below. The proportion of ether repeat units bearing substituent Y is a real number expressed by Equation 1:

$$m / ( n + m ) \qquad Equation 1$$

where m and n each represent a real number between 0 and 1, and the sum of n and m equals 1.

The linear UV-absorbing polyether may for example be a homopolymer or a copolymer optionally containing additional pendant groups other than the UV absorbing chromophore. The distribution of the pendant groups along the polymer chain can be modified to obtain optimal polymer properties. In one embodiment, the linear UV-absorbing polyether is a random copolymer, and the pendant groups including the UV absorbing chromophore are statistically distributed along the polymer chain. In another embodiment, the linear UV-absorbing polyether is a block copolymer, consisting of alternating segments of polymer backbone functionalized with a greater proportion of pendant groups. In another embodiment, the distribution of the pendant groups along the polymer backbone is somewhere between the boundary conditions of block and statistically random copolymers.

The linear UV absorbing polyether comprises one or more covalently bonded UV-chromophores. UV-chromophores that absorb UVA radiation or UVB radiation are suitable. The UV-chromophore may absorb in both the UVA and UVB region.

Suitable UV-chromophores include triazoles (a moiety containing a five-membered heterocyclic ring with two carbon and three nitrogen atoms), such as benzotriazoles including hydroxyphenyl-benzotriazoles, and triazines (a six membered heterocycle containing three nitrogen and three carbon atoms). Examples of other UV-chromophores include camphors such as benzylidene camphor and its derivatives (such as terephthalylidene dicamphor sulfonic acid) and dibenzoylmethanes and their derivatives.

In one embodiment, the UV chromophore is a benzotriazole providing both photostability and strong UVA absorbance with a structure represented in FORMULA II:

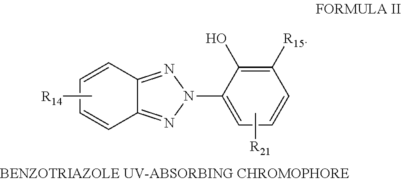

wherein each R₁₄ is independently selected from the group consisting of hydrogen, C₁-C₂₀ alkyl, alkoxy, acyl, alkyloxy, alkylamino, and halogen; R₁₅ is independently selected from the group consisting of hydrogen, C₁-C₂₀ alkyl, alkoxy, acyl, alkyloxy, and alkylamino, R₂₁ is selected from C₁-C₂₀ alkyl, alkoxy, acyl, alkyloxy, and alkylamino. Either of the R₁₅ or R₂₁ groups may include functional groups that allow attachment to a polymer.

Compounds resembling the structure of FORMULA II are described in U.S. Pat. No. 5,869,030, and include, but are not limited to, methylene bis-benzotriazolyl tetramethylbutylphenol.

In one embodiment, the benzotriazole is derived from a transesterification product of 3-(3-(2H-benzo[d][1,2,3]triazol-2-yl)-5-(tert-butyl)-4-hydroxyphenyl) propanoic acid with polyethylene glycol 300, commercially available as TINUVIN 213 from BASF. In another embodiment, the benzotriazole is benzenepropanoic acid, 3-(2H-benzotriazol-2-yl)-5-(1, 1-dimethylethyl)-4-hydroxy-, C₇-9-branched and linear alkyl esters, commercially available as TINUVIN 99 from BASF.

In another embodiment, the UV-absorbing group contains a triazine moiety. An exemplary triazine is 6-octyl-2-(4-(4,6-di([1,1′-biphenyl]-4-yl)-1,3,5-triazin-2-yl)-3-hydroxyphenoxy) propanoate, commercially available as TINUVIN 479 from BASF.

Examples of suitable UVB-absorbing chromophores include 4-aminobenzoic acid and alkane esters thereof; anthranilic acid and alkane esters thereof; salicylic acid and alkane esters thereof; hydroxycinnamic acid alkane esters thereof; dihydroxy, dicarboxy, and hydroxycarboxybenzophenones and alkane ester or acid halide derivatives thereof; dihydroxy, dicarboxy, and hydroxycarboxychalcones and alkane ester or acid halide derivatives thereof; dihydroxy, dicarboxy, and hydroxycarboxycoumarins and alkane ester or acid halide derivatives thereof; benzalmalonate (benzylidene malonate); benzimidazole derivatives (such as phenyl benzilimazole sulfonic acid, PBSA), benzoxazole derivatives, and other suitably functionalized species capable of copolymerization within the polymer chain.

The linear UV-absorbing polyether may include more than one UV-chromophore or more than one type or chemical class of UV-chromophore.

The linear UV absorbing polyether preferably has a low polydispersity. For example, the polydispersity index of the linear UV absorbing polyether may be about 1.5 or less, such as about 1.2 or less. Polydispersity index is defined as Mw/Mn (i.e., the ratio of weight average molecular weight, Mw, to number average molecular weight, Mn).

Linear UV absorbing polyethers may be made as described in U.S. Pat. Nos. 9,255,180 and 9,248,092, the disclosures of which are incorporated in their entirety herein.

A preferred linear UV absorbing polyether is a linear UV absorbing polyether comprising benzotriazole chromophores having the following formula:

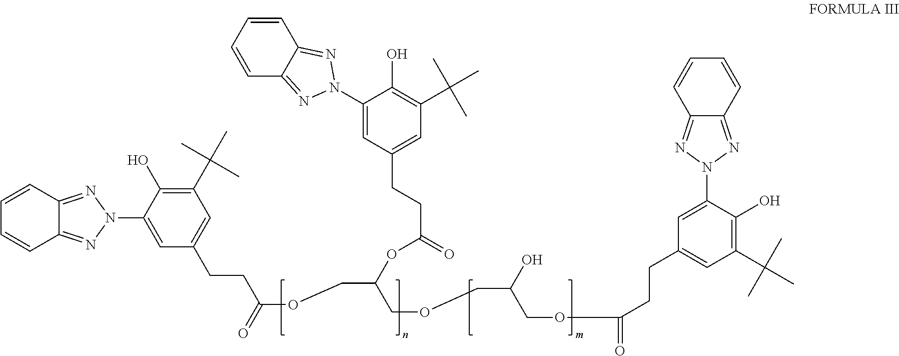

wherein n and m, independently from each other, are each a number from 0 to 20; and wherein at least one of m and n is 1. For example, the sum of n and m is a number from 3 to 10.

For example, such linear UV absorbing polyether comprising benzotriazole chromophores may be based on a polyglycerol backbone containing 5 glycerol units as shown in Formula IV:

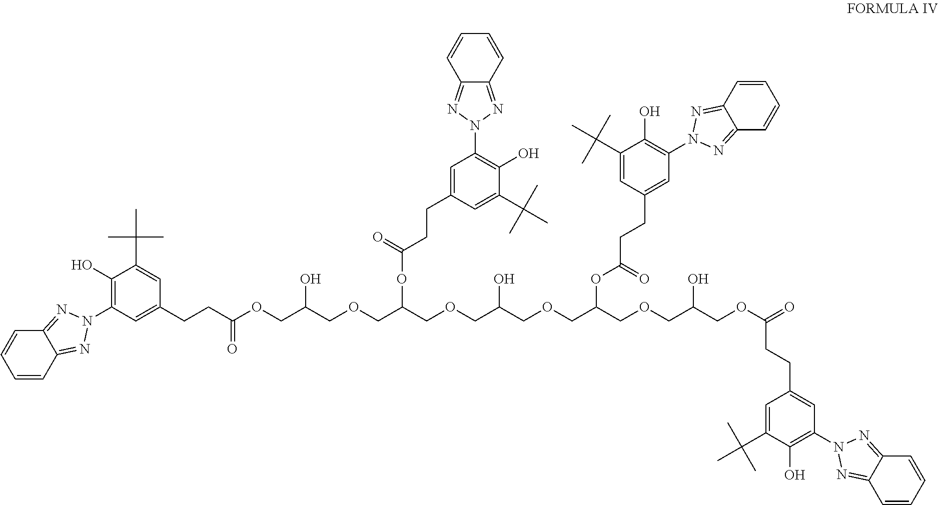 [SMILES: CC(C)(C)c1cc(CCC(=O)OCC(O)COCC(COCC(O)COCC(COCC(O)COC(=O)CCc2cc(-n3nc4ccccc4n3)c(O)c(C(C)(C)C)c2)OC(=O)CCc2cc(-n3nc4ccccc4n3)c(O)c(C(C)(C)C)c2)OC(=O)CCc2cc(-n3nc4ccccc4n3)c(O)c(C(C)(C)C)c2)cc(-n2nc3ccccc3n2)c1O]

The glycerol backbone in these preferred polyethers typically consists mainly of 3 to 10 glycerol units, whereby the hydroxyl groups of the glycerol backbone are covalently linked to the benzotriazole UV chromophore. It might be reasonably assumed that primary hydroxyl groups (terminal units) react faster than secondary hydroxyl groups, which are less reactive for derivatization. Therefore, some secondary hydroxyl groups remain unreacted. The glycerol backbone consists of primarily linear and unbranched structure units. Branched isomers and higher molecular fractions including more than 10 glycerol units can be present.

A particularly preferred linear UV absorbing polyether is polyglyceryl benzotriazolyl t-butylhydroxyphenylpropionate.

Polymer compositions comprising these preferred linear UV absorbing polyethers comprising benzotriazole chromophores may further be characterized as follows:
1. MW distribution: Mn>500 Da, Mw>1200 Da (GPC, calibrated on polystyrene)
2. Benzenepropanoic acid, 3-(2H-benzotriazol-2-yl)-5-(1,1-dimethylethyl)-4-hydroxy-, methyl ester: ≤1.0% (HPLC)
3. Benzenepropanoic acid, 3-(2H-benzotriazol-2-yl)-5-(1,1-dimethylethyl)-4-hydroxy-: ≤1.0% (HPLC)
4. Sum of concentration of Benzenepropanoic acid, 3-(2H-benzotriazol-2-yl)-5-(1,1-dimethyl,ethyl)-4-hydroxy-,methyl ester and Benzenepropanoic acid, 3-(2H-benzotriazol-2-yl)-5-(1,1-dimethylethyl)-4-hydroxy-: ≤1.0% (HPLC)
5. UV-absorption: E 1% 1 cm (344 nm): >310
6. Amount of bound chromophores: >70%
7. Tg (° C.): >50 (DSC)
8. Residual catalyst from transesterification reaction (Tin-II-ethyl hexanoate) <700 ppm or substantially free of Sn (IPC)
9. Solubility in Cetiol B: >30%
10. Solubility in Cetiol AB: >30%

These preferred linear UV-absorbing polyethers comprising benzotriazole chromophores may be prepared by esterification/transesterification reaction, including the steps of reacting a polyglycerol intermediate with a benzotriazole UV-chromophore comprising a complementary functional group to form the polyether, as described in U.S. Ser. No. 10/596,087, the disclosure of which is incorporated in its entirety herein.

The sunscreen composition may contain about 5 to about 20 weight percent, or about 10 to about 15 weight percent, of such a linear UV-absorbing polyether based on the total weight of the sunscreen composition.

### SPF

Sun protection factor (SPF) of the composition may be tested using the following IN VITRO SPF TEST METHOD. The baseline transmission a collagen matrix substrate (in vitro Skin®—IMS—Florida Skincare Testing, Florida USA) is measured for UV absorbance using a calibrated transmission spectrophotometer with integration sphere (Labsphere® UV-1000S UV transmission analyzer, Labsphere® UV-2000S UV transmission analyzer (Labsphere, North Sutton, N.H., USA) or equivalent). A test sample is then applied to the substrate using an application density of about 0.75 mg/cm2, followed by rubbing it into a uniform thin layer with a metallic pointe. The sample is allowed to dry for 15 minutes and then measured for UV absorbance in the same way. The absorbance measures are used to calculate SPF as known in the art using the following equation:

$$SPF in vitro = \frac{{\int }_{λ = 290 nm}^{λ = 400 nm} E ( λ ) * S ( λ ) * d λ}{{\int }_{λ = 290 nm}^{λ = 400 nm} E ( λ ) * S ( λ ) * T ( λ ) * d λ}$$

in which:
E(λ)=Erythema action spectrum;
S(λ)=Spectral irradiance received from the UV source;
T(λ)=Mean transmittance of the test product layer; and
d(λ)=Wavelength step (1 nm).

The sunscreen composition advantageously has a high ratio of SPF to total amount of UV absorbers and UV blockers. In particular, the ratio of the SPF of the composition to the total, combined weight percent of UV absorbers and UV blockers in the composition is at least about 4.1, preferably at least about 4.4.

The sunscreen composition, for example, may have an SPF of at least about 30 as measured by the IN VITRO SPF TEST METHOD wherein the ratio of the SPF of the composition to the total, combined weight percent of UV absorbers and UV blockers in the composition is at least about 4.1.

### Topical Composition

The sunscreen composition can be used by topically applying to a mammal, e.g., by the direct laying on, wiping or spreading of the composition on the skin, hair, or nails of a mammal, particularly a human.

The sunscreen composition preferably comprises a cosmetically acceptable topical carrier. Preferably, the composition is in the form of an oil-in-water emulsion containing a continuous water phase and a discontinuous oil phase dispersed within the continuous water phase. However, other product forms may be employed, such as oil-in-water, oil-water-oil, water-oil-water, or PIT emulsions, microemulsions, anhydrous compositions, gels, oils, creams, milks, lotions, powders, lacquers, tablets, make-up or other color cosmetics, sticks, sprays and aerosols (with or without propellants), foams, pastes, preparations for hair treatment such as shampoos, conditioners, hair-care preparations, e.g., pretreatment preparations, hair tonics, styling creams, styling gels, pomades, hair rinses, treatment packs, intensive hair treatments, hair-straightening preparations, liquid hair-setting preparations, hair foams and hairsprays.

The sunscreen composition may be prepared using mixing and blending methodology well known in the sunscreen and cosmetic arts. In one embodiment, the sunscreen composition is an oil-in-water emulsion produced by preparing an oil phase by mixing the UV absorbers with optional oil soluble or oil-miscible ingredients; and preparing a water phase, by mixing water and optional water-soluble or water-miscible ingredients. The oil phase and the water phase may then be mixed in a manner sufficient to disperse the oil phase substantially homogeneously in the water phase such that the water phase is continuous and the oil phase discontinuous.

In certain embodiments, the sunscreen composition is an oil-in water emulsion that is phase stable.

In certain embodiments, the UV absorbers are dissolved, as opposed to being dispersed or suspended, within the oil phase. Suitable solvents for the UV absorbers include dicaprylyl carbonate available as CETIOL CC from BASF.

The oil phase may be such that it is present in discrete droplets or units having an average diameter of about one micron to about 1000 microns, such as from about 1 micron to about 100 microns.

The percentage by weight of water phase in the composition may range from about 45% to about 90%, such as from about 55% to about 80%, such as from about 60% to about 80%. The percentage by weight of water in the water phase may be about 90% or more, such as about 95% or more.

In certain embodiments, the percentage by weight of oil phase in the composition is from about 10% to about 55%, such as from about 20% to about 45%, such as from about 20% to about 40%.

In certain embodiment, the oil phase consists essentially of the UV absorbers. In certain embodiments, the oil phase contains about 60% or more by weight of the UV absorbers, such as greater than 75% by weight of the UV absorbers, such as greater than 90% by weight of the UV absorbers, such as greater than 97% by weight of the UV absorbers, such as greater than 99% by weight of the UV absorbers.

The composition may be combined with a “cosmetically acceptable topical carrier,” i.e., a carrier for topical use capable of containing the other ingredients dispersed or dissolved therein and possessing acceptable properties rendering it safe to use topically.

The composition may optionally comprise a wide variety of additional oil-soluble materials and/or water-soluble materials conventionally used in compositions for use on skin or hair, at their art-established levels. For example, emulsifiers, surfactants, pearlescent or opacifying agents, thickeners, emollients, conditioners, humectants, chelating agents, exfoliants, and additives that enhance the appearance, feel, or fragrance of the cleansing composition, such as colorants, fragrances, preservatives, pH adjusting agents, and the like, can be included.

Water soluble or water dispersible polymers may be added to the compositions. Water dispersible polymers are comprised of a water-insoluble polymer that is typically micronized and dispersed into a water carrier, possibly with the use of a surface active dispersing aid. The water dispersible polymers are capable of forming a film and improving water resistance of the compositions.

Examples of water soluble polymers include Polyaldo® 10-1-L (Polyglyceryl-10 Laurate), available from Lonza. Examples of water dispersible polymers include water dispersible polyurethanes, such as Baycusan® C1000 (Polyurethane-34), available from Bayer, Dow Corning® 2501 (Bis-PEG-18 Methyl Ether Dimethyl Silane), available from Dow Corning, Eastman AQ™ 38S (Polyester-5), available from Eastman Chemical, and Intelimer® 8600 (C8-22 Alkyl Acrylates/Methacrylic Acid Crosspolymer) available from Air Products.

The sunscreen composition may also comprise a film former. Film formers are typically hydrophobic materials that impart film forming and/or waterproofing characteristics. One such agent is polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Another suitable film former is synthetic wax, also available from New Phase Technologies as Performa® V-825. Other typical film-formers include acrylates/acrylamide copolymer, acrylates copolymer, acrylates/C12-C22 alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis fordi Oil copolymer, decamethyl cyclopentasiloxane (and) trimethylsiloxysilicate, Dimethicone; Acrylates/Dimethicone Copolymer, hydrolyzed jojoba esters; jojoba esters and mixtures thereof. In some cases, the film former is acrylates/C12-C22 alkylmethacrylate copolymer sold under the tradename Allianz™ OPT by Ashland.

Preferably, the film former is selected from the group consisting of hydrolyzed jojoba esters, jojoba esters, triacontanyl PVP, acrylates/dimethicone copolymer, and mixtures thereof.

Suitable emollients include mineral oils, petrolatum, vegetable oils (e.g., triglycerides such as caprylic/capric triglyceride), waxes and other mixtures of fatty esters, including but not limited to isopropyl palmitate, isopropyl myristate, diisopropyl adipate, dicaprylyl carbonate, C12-15 alkyl benzoate, diisopropyl sebacate, isononyl isononanoate and silicone oils such as dimethicone. Preferably, the emollients are selected from the group consisting of C12-15 alkyl benzoate, diisopropyl adipate, diisopropyl sebacate, isopropyl palmitate and isononyl isononanoate, and mixtures thereof.

In certain embodiments, mixtures of triglycerides (e.g., caprylic/capric triclycerides) and esters of glycols may be used to solubilize the UV absorbers. In other embodiments of the invention, the compositions are substantially free of, or free of, emollients.

Other examples of suitable solvents include propylene glycol, polyethylene glycol, polypropylene glycol, glycerol, 1,2,4-butanetriol, sorbitol esters, 1,2,6-hexanetriol, ethanol, and mixtures thereof.

The composition may comprise an emulsifier. Preferably the emulsifier is selected from the group consisting of glyceryl stearate; PEG-100 stearate, cetyl alcohol; PEG-75 stearate; ceteth-20; steareth-20, potassium cetyl phosphate, and mixtures thereof.

In certain embodiments, the composition includes a pigment suitable for providing color or hiding power. The pigment may be one suitable for use in a color cosmetic product, including compositions for application to the hair, nails and/or skin, especially the face. Color cosmetic compositions include, but are not limited to, foundations, concealers, primers, blush, mascara, eyeshadow, eyeliner, lipstick, nail polish and tinted moisturizers. The pigment suitable for providing color or hiding power may be composed of iron oxides, including red and yellow iron oxides, titanium dioxide, ultramarine and chromium or chromium hydroxide colors, and mixtures thereof. The pigment may be a lake pigment, e.g., an organic dye such as azo, indigoid, triphenylmethane, anthraquinone, and xanthine dyes that are designated as D&C and FD&C blues, browns, greens, oranges, reds, yellows, etc., precipitated onto inert binders such as insoluble salts. Examples of lake pigments include Red #6, Red #7, Yellow #5, Violet #2 and Blue #1. The pigment may be an interference pigment. Examples of interference pigments include those containing mica substrates, bismuth oxycloride substrates, and silica substrates, for instance mica/bismuth oxychloride/iron oxide pigments commercially available as CHROMALITE pigments (BASF), titanium dioxide and/or iron oxides coated onto mica such as commercially available FLAMENCO pigments (BASF), mica/titanium dioxide/iron oxide pigments including commercially available KTZ pigments (Kobo products), CELLINI pearl pigments (BASF), and borosilicate-containing pigments such as REFLECKS pigments (BASF).

In certain embodiments, the sunscreen composition may include one or more compounds suitable for enhancing the photostability of the UV absorbers or other ingredients in the composition. Photostabilizers include, for example, diesters or polyesters of a naphthalene dicarboxylic acid.

In one embodiment, the sunscreen composition comprises a humectant. Examples of humectants include, without limitation to, glycerin, pentylene glycol, butylene glycol.

In one embodiment, the sunscreen composition comprises a humectant selected from the group consisting of glycerin, pentylene glycol, and mixtures thereof.

The sunscreen composition may advantageously comprise about 0.5 to about 2 weight percent glycerin. The sunscreen composition may comprise for example about 1 weight percent of glycerin.

In another embodiment, the sunscreen composition has a pH of about 5 to about 6. The sunscreen composition may have a pH of about 5.5.

The composition may comprise one or more other cosmetically acceptable active agents include for example anti-acne agents, shine control agents, anti-microbial agents, anti-inflammatory agents, anti-mycotic agents, anti-parasite agents, external analgesics, sunscreens, photoprotectors, antioxidants, keratolytic agents, surfactants, moisturizers, nutrients, vitamins, energy enhancers, anti-perspiration agents, astringents, deodorants, firming agents, anti-callous agents, and agents for skin or hair conditioning.

The amount of other cosmetically active agent in may range from about 0.001% to about 20% by weight of the composition, e.g., about 0.005% to about 10% by weight of the composition, such as about 0.01% to about 5% by weight of the composition.

The cosmetically acceptable active agent may be selected for instance from benzoyl peroxide, D-panthenol carotenoids, ceramides, polyunsaturated fatty acids, essential fatty acids, enzymes such as laccase, enzyme inhibitors, minerals, hormones such as estrogens, steroids such as hydrocortisone, 2-dimethylaminoethanol, copper salts such as copper chloride, peptides like argireline, syn-ake, those containing copper, and acetyl dipeptide-31 amide, coenzyme Q10, amino acids such as proline, vitamins, lactobionic acid, acetyl-coenzyme A, niacin, riboflavin, thiamin, ribose, electron transporters such as NADH and FADH2, natural extracts such as from aloe vera, feverfew, oatmeal, dill, blackberry, princess tree, Picia anomala, and chicory, resorcinols such as 4-hexyl resorcinol, curcuminoids, sugar amines such as acetyl glucosamines, glycosaminoglycans such as hyaluronic acid or its sodium salt (sodium hyaluronate), and derivatives and mixtures thereof.

The composition may advantageously comprise about 0.5 to about 2 weight percent of feverfew extract, for example about 1 weight percent of feverfew extract.

The composition may advantageously comprise about 2 to about 6 weight percent of acetyl glucosamine, for example about 4 weight percent of acetyl glucosamine.

The composition may advantageously comprise about 0.01 to about 0.1 weight percent of sodium hyaluronate, for example about 0.05 weight percent of sodium hyaluronate.

Examples of vitamins include, but are not limited to, vitamin A, vitamin B's such as vitamin B3, vitamin B5, and vitamin B12, vitamin C, vitamin K, and different forms of vitamin E like alpha, beta, gamma or delta tocopherols or their mixtures, and derivatives thereof.

The composition may advantageously comprise about 1 to about 5 weight percent of niacinamide, for example about 2 weight percent of niacinamide.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate, ascorbyl polypeptide, and sodium ascorbyl phosphate). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol, rutin, and the like. Examples of such natural extracts include grape seed, green tea, pine bark, and propolis.

The composition may comprise about 0.1 to about 0.5 weight percent of tocopherol acetate, for example about 0.2 weight percent of tocopherol acetate.

The composition may comprise about 0.1 to about 0.5 weight percent of sodium ascorbyl phosphate, for example about 0.2 weight percent of sodium ascorbyl phosphate.

Advantageously, the composition may comprise a cosmetically acceptable active agent selected from the group consisting of niacinamide, tocopheryl acetate, sodium ascorbyl phosphate, acetyl glucosamine, trehalose, feverfew extract, glycerin, sodium hyaluronate and capryloyl glycine (and) sarcosine (and) Cinnamomum Seylanicum Bark Extract, and Mixtures Thereof.

For example, the composition may comprise a cosmetically acceptable active agent selected from the group consisting of niacinamide, glycerin, sodium hyaluronate, and mixtures thereof.

The composition may comprise about 1 to about 5 weight percent of niacinamide, about 0.5 to about 2 weight percent glycerin, and about 0.01 to about 0.1 weight percent of sodium hyaluronate.

The composition may comprise about 2 weight percent of niacinamide, about 1 weight percent glycerin, and about 0.05 weight percent of sodium hyaluronate.

Alternatively, the composition may comprise a cosmetically acceptable active agent selected from the group consisting of acetyl glucosamine, niacinamide, and mixtures thereof.

For example, the composition may comprise about 2 to about 6 weight percent of acetyl glucosamine and about 1 to about 5 weight percent of niacinamide.

The composition may comprise about 4 weight percent of acetyl glucosamine, and about 4 weight percent of niacinamide.

The following non-limiting examples further illustrate the invention. The described compositions were generally prepared as follows.

For formulas that did not contain pigments: Liposoluble ingredients were mixed together as the “oil phase” in a main tank/pot and heated to 75-85° C. to assure proper solubility of all ingredients. After all ingredients were soluble, dispersed materials such as viscosity increasing agents were added. The phase was stirred and homogenized to assure proper dispersion of all materials and then hot water (75-85° C.) was poured into the main tank with oil phase under high sheer and homogenization to promote emulsification. After an emulsion was formed, the mixture was cooled to 60° C. or below and temperature sensitive ingredients such as MBBT were added while stirring. In the next step, the mixture was cooled to 35-40° C. and other temperature sensitive ingredients such as fragrance and antioxidants were added while stirring. Finally, the mixture was homogenized and cooled to 25° C. for pH measurement and adjustment with sodium hydroxide if needed.

For formulas containing pigments: Hot water and pigments were heated to 75-85° C. in a main tank with a chelating agent and homogenized at a high sheer to promote pigment opening. Liposoluble ingredients were mixed together as the “oil phase” in a second tank/pot and heated to 75-85° C. to assure proper solubility of all ingredients. After all ingredients were soluble, dispersed materials such as viscosity increasing agents were added. The phase was stirred and homogenized to assure proper dispersion of all materials. With both phases at 75-85° C. the oil phase was poured into the water under high sheer and homogenization to promote emulsification. After an emulsion was formed, the mixture was cooled to 60° C. or below and temperature sensitive ingredients such as MBBT were added while stirring. In the next step, the mixture was cooled to 35-40° C. and other temperature sensitive ingredients such as fragrance and antioxidants were added while stirring. Finally, the mixture was homogenized and cooled to 25° C. for pH measurement and adjustment with sodium hydroxide if needed.

### Example 1

A series of compositions were prepared and tested for SPF using the IN VITRO SPF TEST METHOD. The compositions were formulated in the vehicles shown in Table 1 and contained the UV absorbers and blockers shown in Table 2. For each composition the ratio of SPF to total weight percent of all UV absorbers and UV blockers was calculated. The results are also shown in Table 2.

TABLE 1

| | | Composition | | | | | | | |
|---|---|---|---|---|---|---|---|---|---|
| INCI Name | Function | 1 | 2 | 3 | 4 | 5 | 6 | 7 | 8 |
| Triacontanyl PVP | FILM FORMING | 0.0 | 0.0 | 0.0 | 0.7 | 0.7 | 0.7 | 0.7 | 0.7 |
| | AGENT | | | | | | | | |
| Aluminum Starch | ABSORBENT | 2.0 | 2.0 | 2.0 | 2.0 | 2.0 | 2.0 | 2.0 | 2.0 |
| Octenylsuccinate | | | | | | | | | |
| Phenoxyethanol | PRESERVATIVE | 0.56 | 0.8 | 0.8 | 0.8 | 0.8 | 0.8 | 0.8 | 0.8 |
| Acrylates/C10-30 Alkyl | VISCOSITY | 0.23 | 0.24 | 0.32 | 0.27 | 0.23 | 0.23 | 0.23 | 0.23 |
| Acrylate Crosspolymer | INCREASING | | | | | | | | |
| C12-15 Alkyl Benzoate | EMOLLIENT | 0.0 | 0.0 | 0.0 | 0.0 | 11.0 | 7.0 | 7.0 | 7.0 |
| Disodium EDTA | CHELATING | 0.1 | 0.1 | 0.1 | 0.1 | 0.1 | 0.1 | 0.1 | 0.1 |
| | AGENT | | | | | | | | |
| Tocopheryl Acetate | ANTIOXIDANT | 0.1 | 0.5 | 0.1 | 0.50 | 0.0 | 0.0 | 0.0 | 0.0 |
| Sodium Hydroxide | PH ADJUSTER | 0.03 | 0.03 | 0.03 | 0.03 | 0.03 | 0.03 | 0.03 | 0.03 |
| Potassium Cetyl | EMULSIFIER | 0.5 | 1.0 | 1.0 | 1.0 | 1.0 | 1.0 | 1.0 | 1.0 |
| Phosphate | | | | | | | | | |
| Water | SOLVENT | 74.18 | 62.53 | 61.35 | 69.1 | 58.7 | 58.7 | 58.7 | 58.7 |
| Cetyl Alcohol; Glyceryl | SURFACTANT | 2.0 | 2.0 | 0.0 | 2.0 | 2.0 | 2.0 | 2.0 | 2.0 |
| Stearate; PEG-75 | | | | | | | | | |
| Stearate; Ceteth-20; | | | | | | | | | |
| Steareth-20 | | | | | | | | | |
| Silica | ABSORBENT | 0.0 | 0.3 | 1.0 | 0.3 | 0.3 | 0.3 | 0.3 | 0.3 |
| Diisopropyl Adipate | EMOLLIENT | 0.2 | 1.0 | 1.0 | 1.0 | 10.0 | 7.5 | 7.5 | 7.5 |
| Glycerin | HUMECTANT | 2.0 | 2.0 | 2.0 | 2.0 | 2.0 | 2.0 | 2.0 | 2.0 |
| Dimethicone; | FILM FORMING | 0.0 | 0.5 | 0.5 | 0.5 | 0.5 | 0.5 | 0.5 | 0.5 |
| Acrylates/Dimethicone | AGENT | | | | | | | | |
| Copolymer | | | | | | | | | |
| Hydroxyacetophenone | SKIN | 0.6 | 0.6 | 0.6 | 0.6 | 0.6 | 0.6 | 0.6 | 0.6 |
| | CONDITIONER | | | | | | | | |
| Sodium Potassium | BULKING AGENT | 0.0 | 1.25 | 1.5 | 1.0 | 1.5 | 1.5 | 1.5 | 1.5 |
| Aluminum Silicate; | | | | | | | | | |
| Titanium Dioxide; Silica | | | | | | | | | |
| Hydrolyzed Jojoba | EMOLLIENT | 0.5 | 0.5 | 0.5 | 0.5 | 0.5 | 0.5 | 0.5 | 0.5 |
| Esters; Jojoba Esters; | | | | | | | | | |
| Water | | | | | | | | | |
| Fragrance | FRAGRANCE | 0.25 | 0.35 | 0.2 | 0.35 | 0.0 | 0.0 | 0.0 | 0.0 |
| Glyceryl Stearate; | EMULSIFIER | 0.0 | 0.0 | 1.0 | 0.0 | 0.0 | 0.0 | 0.0 | 0.0 |
| PEG-100 Stearate | | | | | | | | | |
| Sodium Polyacrylate | VISCOSITY | 0.2 | 0.0 | 0.0 | 0.0 | 0.0 | 0.0 | 0.0 | 0.0 |
| | INCREASING | | | | | | | | |

TABLE 2
UV

| Absorber/Blocker | Composition | | | | | | | |
|---|---|---|---|---|---|---|---|---|
| (Filter) | 1 | 2 | 3 | 4 | 5 | 6 | 7 | 8 |
| Avobenzone (wt %) | 3 | 3.8 | 4 | 3 | 0 | 0 | 0 | 0 |
| Octocrylene (wt %) | 4.5 | 4.5 | 4.5 | 3.5 | 0 | 0 | 0 | 0 |
| Octisalate (wt %) | 0 | 2.6 | 4.5 | 0 | 0 | 0 | 0 | 0 |
| Homosalate (wt %) | 0.2 | 0.5 | 1 | 5 | 0 | 0 | 0 | 0 |
| BEMT (wt %) | 0.2 | 0.5 | 1 | 0.5 | 1 | 1.5 | 1 | 1 |
| EHT (wt %) | 1 | 1 | 2.5 | 1 | 2 | 2 | 3 | 3 |
| TiO₂ (wt %) | 0 | 0.3 | 0.3 | 0.3 | 8 | 5 | 5 | 1 |
| MBBT (wt %) | 3 | 5 | 4.5 | 4 | 7 | 5 | 6 | 5 |
| DHHB | 0 | 0 | 0 | 0 | 0 | 1 | 0 | 3 |
| TBT | 0 | 0 | 0 | 0 | 3 | 5 | 0 | 1.5 |
| PBSA | 0 | 0 | 0 | 0 | 0 | 2 | 0 | 0 |
| ZnO | 0 | 0 | 0 | 0 | 0 | 0 | 7 | 0 |
| Total (wt %) | 17.7 | 23.6 | 25.3 | 16.30 | 21 | 21.5 | 22 | 14.5 |
| SPF | 16.03 | 86.32 | 87.17 | 26.99 | 64.77 | 83.43 | 20.87 | 95.71 |
| SPF/wt % of Filters | 0.91 | 3.66 | 3.45 | 1.65 | 3.1 | 3.9 | 0.9 | 6.6 |

All of the compositions contained the high molecular weight UV absorbers BEMT, EHT, and MBBT. However, Compositions 1-4 also contained one or more of the low molecular weight UV absorbers avobenzone, octocrylene, octisalate, and homosalate. Compositions 1-4 had a relatively low ratio of SPF to total weight percent of filters (UV absorbers plus UV blockers), the highest of which was 3.66 (Composition 2). This indicated a low filter pack efficiency. Composition 5 contained no low molecular weight UV absorbers but did not contain DHHB and provided an SPF of 64.77 and a ratio of SPF to total weight percent of filters of only 3.1. Composition 6 contained the low molecular weight UV absorber PBSA and delivered a ratio of SPF to total weight percent of filters of 3.9. Composition 7 contained no low molecular weight UV absorbers but did not contain DHHB and provided an SPF of only 20.87 and a ratio of SPF to total weight percent of filters of only 0.9. Composition 8 contained no low molecular weight UV absorbers and each of BEMT, EHT, MBBT, DHHB, TiO2, and TBT. It provided the highest SPF of 95.71 and the highest a ratio of SPF to total weight percent of filters of 6.6.

### Example 2

A series of compositions were prepared and tested for SPF using the IN VITRO SPF TEST METHOD. The compositions were formulated in the vehicles shown in Table 3 and contained the UV absorbers and blockers shown in Table 4. For each composition the ratio of SPF to total weight percent of all UV absorbers and UV blockers was calculated. The results are also shown in Table 4.

TABLE 3

| | | Composition | | | | | | | | | |
|---|---|---|---|---|---|---|---|---|---|---|---|
| INCI Name | Function | 9 | 10 | 11 | 12 | 13 | 14 | 15 | 16 | 17 | 18 |
| Triacontanyl PVP | FILM FORMING | 0.35 | 0.70 | 1.70 | 0.35 | 0.70 | 1.70 | 0.35 | 1.00 | 1.00 | 0.35 |
| | AGENT | | | | | | | | | | |
| Aluminum Starch | ABSORBENT | 2.00 | 2.00 | 3.00 | 2.50 | 2.00 | 3.00 | 2.00 | 3.00 | 3.00 | 2.50 |
| Octenylsuccinate | | | | | | | | | | | |
| Phenoxyethanol | PRESERVATIVE | 0.56 | 0.80 | 0.80 | 0.00 | 0.80 | 0.00 | 0.00 | 0.00 | 0.00 | 0.00 |
| Acrylates/C10-30 Alkyl | VISCOSITY | 0.20 | 0.23 | 0.18 | 0.15 | 0.23 | 0.15 | 0.20 | 0.00 | 0.00 | 0.15 |
| Acrylate Crosspolymer | INCREASING | | | | | | | | | | |
| Disodium EDTA | CHELATING | 0.10 | 0.10 | 0.10 | 0.20 | 0.10 | 0.10 | 0.10 | 0.10 | 0.10 | 0.10 |
| | AGENT | | | | | | | | | | |
| Isopropyl Palmitate | EMOLLIENT | 6.40 | 0.00 | 0.00 | 0.00 | 0.00 | 0.00 | 0.00 | 0.00 | 0.00 | 0.00 |
| Stearyl Alcohol | EMULSIFIER | 0.00 | 0.00 | 0.00 | 0.00 | 0.00 | 0.50 | 1.50 | 0.00 | 0.00 | 0.00 |
| Sodium Hydroxide | PH ADJUSTER | 0.05 | 0.03 | 0.05 | 0.00 | 0.03 | 0.05 | 0.05 | 0.00 | 0.00 | 0.00 |
| Potassium Cetyl | EMULSIFIER | 0.50 | 1.00 | 1.00 | 0.50 | 1.00 | 1.80 | 1.20 | 0.00 | 0.00 | 0.50 |
| Phosphate | | | | | | | | | | | |
| Sodium Ascorbyl | ANTIOXIDANT | 0.00 | 0.00 | 0.20 | 0.20 | 0.00 | 0.20 | 0.00 | 0.20 | 0.20 | 0.20 |
| Phosphate | | | | | | | | | | | |
| Isononyl Isononanoate | EMOLLIENT | 0.00 | 0.00 | 0.00 | 0.00 | 0.00 | 0.00 | 0.00 | 4.00 | 4.00 | 0.00 |
| Glyceryl Stearate; | EMULSIFIER | 0.00 | 0.00 | 2.00 | 1.50 | 0.00 | 0.00 | 0.00 | 0.00 | 0.00 | 1.50 |
| PEG-100 Stearate | | | | | | | | | | | |
| Silica | ABSORBENT | 0.00 | 0.30 | 0.00 | 0.00 | 0.30 | 6.00 | 1.00 | 5.00 | 3.70 | 0.00 |
| Water | SOLVENT | 70.74 | 57.74 | 46.17 | 63.95 | 53.74 | 39.10 | 39.31 | 51.15 | 52.05 | 64.25 |
| Caprylyl Methicone | SKIN COND | 0.00 | 0.00 | 1.50 | 3.00 | 0.00 | 1.50 | 2.00 | 1.00 | 1.00 | 3.00 |
| | OCCLUSIVE | | | | | | | | | | |
| Sodium Polyacrylate | VISCOSITY | 0.00 | 0.00 | 0.00 | 0.00 | 0.00 | 0.00 | 0.20 | 0.00 | 0.00 | 0.00 |
| | INCREASING | | | | | | | | | | |
| Cetyl Alcohol; Glyceryl | EMULSIFIER | 2.00 | 2.00 | 0.00 | 0.00 | 2.00 | 0.00 | 2.00 | 0.40 | 0.40 | 0.00 |
| Stearate; PEG-75 Stearate; | | | | | | | | | | | |
| Ceteth-20; Steareth-20 | | | | | | | | | | | |
| Diisopropyl Sebacate | EMOLLIENT | 0.00 | 0.00 | 0.00 | 1.00 | 0.00 | 3.50 | 2.10 | 2.00 | 3.00 | 1.00 |
| Hydroxyethyl | VISCOSITY | 0.00 | 0.00 | 0.00 | 0.30 | 0.00 | 0.00 | 0.00 | 0.30 | 0.50 | 0.30 |
| Acrylate/Sodium | INCREASING | | | | | | | | | | |
| Acryloyldimethyl | | | | | | | | | | | |
| Taurate Copolymer | | | | | | | | | | | |
| Diisopropyl Adipate | EMOLLIENT | 0.00 | 10.00 | 7.00 | 3.00 | 10.00 | 3.50 | 2.60 | 2.00 | 2.50 | 3.00 |
| Glycerin | HUMECTANT | 2.00 | 2.00 | 0.00 | 1.00 | 2.00 | 0.00 | 2.00 | 5.00 | 3.00 | 1.00 |
| Trehalose | SKIN | 0.00 | 0.00 | 0.00 | 0.00 | 0.00 | 2.00 | 0.00 | 0.00 | 0.00 | 0.00 |
| | CONDITIONER | | | | | | | | | | |
| Caprylyl Glycol; | HUMECTANT | 0.00 | 0.00 | 0.00 | 0.00 | 0.00 | 0.00 | 0.00 | 0.00 | 0.80 | 0.00 |
| Caprylhydroxamic | | | | | | | | | | | |
| Acid; Glycerin | | | | | | | | | | | |
| Coco-Caprylate | EMOLLIENT | 1.20 | 0.00 | 0.00 | 0.00 | 0.00 | 0.00 | 0.00 | 0.00 | 0.00 | 0.00 |
| Tocopheryl Acetate | HAIR | 0.10 | 0.00 | 0.10 | 0.20 | 0.00 | 0.10 | 0.00 | 0.20 | 0.00 | 0.20 |
| | CONDITIONER | | | | | | | | | | |
| Glyceryl Stearate | EMULSIFIER | 0.00 | 0.00 | 0.00 | 0.00 | 0.00 | 0.00 | 0.00 | 0.00 | 0.30 | 0.00 |
| Iron Oxides; Sodium | COLORANT | 0.00 | 0.00 | 0.00 | 0.00 | 0.00 | 0.00 | 0.75 | 0.00 | 0.00 | 0.00 |
| C14-16 Olefin Sulfonate | | | | | | | | | | | |
| Chrysanthemum | SKIN | 0.00 | 0.00 | 0.30 | 0.00 | 0.00 | 0.30 | 1.00 | 0.00 | 0.30 | 0.00 |
| Parthenium (Feverfew) | CONDITIONER | | | | | | | | | | |
| Flower/Leaf/Stem Juice | | | | | | | | | | | |
| Titanium Dioxide; Sodium | COLORANT | 0.00 | 0.00 | 0.00 | 0.00 | 0.00 | 0.00 | 5.16 | 0.00 | 0.00 | 0.00 |
| C14-16 Olefin Sulfonate | | | | | | | | | | | |
| Iron Oxides; Sodium C14-16 | COLORANT | 0.00 | 0.00 | 0.00 | 0.00 | 0.00 | 0.00 | 0.03 | 0.00 | 0.00 | 0.00 |
| Olefin Sulfonate (black) | | | | | | | | | | | |
| Iron Oxides; Sodium C14-16 | COLORANT | 0.00 | 0.00 | 0.00 | 0.00 | 0.00 | 0.00 | 0.15 | 0.00 | 0.00 | 0.00 |
| Olefin Sulfonate (red) | | | | | | | | | | | |
| Dimethicone; | EMOLLIENT | 0.00 | 0.50 | 0.00 | 0.00 | 0.50 | 0.00 | 0.00 | 0.00 | 0.00 | 0.00 |
| Acrylates/Dimethicone | | | | | | | | | | | |
| Copolymer | | | | | | | | | | | |
| Cetearyl Alcohol; | SURFACTANT | 0.00 | 0.00 | 0.00 | 0.00 | 0.00 | 1.80 | 0.00 | 0.00 | 0.00 | 0.00 |
| Coco-Glucoside | | | | | | | | | | | |
| C12-15 Alkyl Benzoate | SOLUBILIZER | | 6.50 | 8.00 | 4.00 | 6.50 | 8.00 | 9.80 | 4.50 | 5.00 | 4.00 |
| Sodium | VISCOSITY | 0.00 | 0.00 | 0.00 | 0.00 | 0.00 | 0.00 | 0.00 | 0.00 | 0.20 | 0.00 |
| Acryloyldimethyltaurate/VP | INCREASING | | | | | | | | | | |
| Crosspolymer | | | | | | | | | | | |
| Sodium Potassium | BULKING | 0.00 | 1.50 | 1.50 | 0.00 | 1.50 | 0.00 | 0.00 | 0.00 | 0.00 | 0.00 |
| Aluminum Silicate; | AGENT | | | | | | | | | | |
| Titanium Dioxide; Silica | | | | | | | | | | | |
| Sodium Hyaluronate | HUMECTANT | 0.00 | 0.00 | 0.00 | 0.05 | 0.00 | 0.00 | 0.00 | 0.05 | 0.05 | 0.05 |
| Panthenol | HAIR | 0.00 | 0.00 | 0.00 | 0.10 | 0.00 | 0.00 | 0.00 | 0.10 | 0.10 | 0.10 |
| | CONDITIONER | | | | | | | | | | |
| Hydrolyzed Jojoba Esters; | EMOLLIENT | 0.50 | 0.50 | 0.00 | 0.30 | 0.50 | 0.00 | 0.50 | 0.00 | 0.00 | 0.30 |
| Jojoba Esters; Water | | | | | | | | | | | |
| Dimethicone; Dimethicone | SKIN | 0.00 | 0.00 | 2.00 | 2.00 | 0.00 | 2.00 | 1.50 | 2.00 | 1.00 | 2.00 |
| Crosspolymer | CONDITIONER | | | | | | | | | | |
| Hydroxyacetophenone | SKIN | 0.60 | 0.60 | 0.60 | 0.70 | 0.60 | 0.60 | 0.60 | 0.60 | 0.80 | 0.60 |
| | CONDITIONER | | | | | | | | | | |
| Acetyl Glucosamine | SKIN | 0.00 | 0.00 | 0.00 | 0.00 | 0.00 | 0.00 | 4.00 | 0.00 | 0.00 | 0.00 |
| | CONDITIONER | | | | | | | | | | |
| Cocoglycerides | EMOLLIENT | 3.20 | 0.00 | 0.00 | 0.00 | 0.00 | 0.00 | 0.00 | 0.00 | 0.00 | 0.00 |
| Niacinamide | SKIN | 0.00 | 0.00 | 1.00 | 2.00 | 0.00 | 2.00 | 4.00 | 2.00 | 1.00 | 2.00 |
| | CONDITIONER | | | | | | | | | | |
| Pentylene Glycol | SKIN | 0.00 | 0.00 | 0.00 | 0.10 | 0.00 | 1.00 | 0.00 | 0.00 | 0.00 | 0.00 |
| | CONDITIONER | | | | | | | | | | |
| Alcohol | SOLVENT | 0.00 | 0.00 | 2.00 | 0.00 | 0.00 | 2.00 | 0.00 | 3.00 | 4.00 | 0.00 |
| Silica (rice) | ABRASIVE | 0.00 | 0.00 | 3.00 | 0.00 | 0.00 | 0.00 | 0.00 | 0.00 | 0.00 | 0.00 |
| Polyacrylate | VISCOSITY | 0.00 | 0.00 | 0.00 | 0.00 | 0.00 | 0.00 | 0.00 | 0.00 | 0.00 | 0.00 |
| Crosspolymer-6 | INCREASING | | | | | | | | | | |
| Fragrance | FRAGRANCE | 0.00 | 0.00 | 0.00 | 0.10 | 0.00 | 0.00 | 0.30 | 0.10 | 0.00 | 0.10 |

TABLE 4
UV

| Absorber/Blocker | Composition | | | | | | | | | |
|---|---|---|---|---|---|---|---|---|---|---|
| (Filter) | 9 | 10 | 11 | 12 | 13 | 14 | 15 | 16 | 17 | 18 |
| DHHB (wt %) | 2 | 2.45 | 6 | 4 | 3 | 5.5 | 4.5 | 4 | 4.5 | 4 |
| TBPT (wt %) | 0 | 2.45 | 1 | 0 | 0 | 1 | 0 | 0 | 0 | 0 |
| BEMT (wt %) | 0.5 | 2 | 2 | 0.5 | 1.5 | 2.3 | 1.8 | 0.5 | 3.5 | 0.5 |
| EHT (wt %) | 2.5 | 4.5 | 4.5 | 4 | 2 | 4.5 | 4 | 4 | 4 | 4 |
| TiO₂ (wt %) | 0.5 | 1.64 | 0.3 | 0.3 | 2 | 0.3 | 0.3 | 0.3 | 0 | 0.3 |
| MBBT (wt %) | 4 | 4.09 | 4 | 4 | 6 | 5.5 | 5 | 4 | 0 | 4 |
| Total (wt %) | 9.5 | 13.5 | 17.8 | 12.8 | 17.5 | 19.1 | 15.6 | 12.2 | 12 | 12.8 |
| SPF | 52.23 | 57.97 | 96.44 | 59.35 | 83.52 | 103.26 | 92.48 | 78.68 | 52.63 | 64.63 |
| SPF/wt % of | 5.5 | 4.29 | 5.42 | 4.64 | 4.77 | 5.41 | 5.93 | 6.45 | 4.38 | 5.05 |
| Filters | | | | | | | | | | |

Compositions 9-16 and 18 each contained the combination of UV absorbers DHHB, BEMT, EHT and MBBT according to the invention and all provided high SPF values of 52.23 to 103.26. In addition, these Compositions all provided high filter pack efficiencies, having ratios of SPF to total weight percent of filters of 4.29 to 6.45. While Composition 17 did not contain MBBT, it did provide an SPF of 52.63 and a ratio of SPF to total weight percent of filters of 4.38. It is believed this is because it contained EHT and BEMT in high concentrations and these UV absorbers have a higher UVB absorbance than MBBT. However, when Composition 17 was tested for SPF in vivo, the result was 44, giving a ratio of SPF to total weight percent of filters of only 3.66. In contrast, Compositions 15 and 16 provided in vivo SPF results of 72 and 74, with ratios of SPF to total weight percent of filters of 4.61 and 6.06, respectively, which were much higher than those of Composition 17.

### Example 3

A series of compositions according to the invention were made using the following UV absorbers and blockers in the following amounts: BEMT 0.5 wt %, EHT 2.5 wt %, MBBT 4 wt %, DHHB 2 wt %, and TiO2 0.5 wt %. The compositions also contained 0-20 wt % of the polymeric UV absorber polyglyceryl benzotriazolyl t-butylhydroxyphenylpropionate. The compositions were formulated using the vehicle shown in Table 5.

TABLE 5

| | | Weight |
|---|---|---|
| Chemical Name | Function | Percent |
| Triacontanyl PVP | FILM FORMING AGENT | 0.3 |
| Aluminum Starch Octenylsuccinate | ABSORBENT | 2.0 |
| Phenoxyethanol | PRESERVATIVE | 0.56 |
| Acrylates/C10-30 Alkyl | VISCOSITY INCREASING | 0.2 |
| Acrylate Crosspolymer | | |
| Disodium EDTA | CHELATING AGENT | 0.1 |
| Isopropyl Palmitate | EMOLLIENT | 6.4 |
| Tocopheryl Acetate | ANTIOXIDANT | 0.1 |
| Sodium Hydroxide | PH ADJUSTER | 0.05 |
| Potassium Cetyl Phosphate | EMULSIFIER | 0.5 |
| Water | SOLVENT | 70.74 |
| | | qs |
| Cetyl Alcohol; Glyceryl | EMULSIFIER | 2.0 |
| Stearate; PEG-75 Stearate; | | |
| Ceteth-20; Steareth-20 | | |
| Glycerin | HUMECTANT | 2.0 |
| Coco-Caprylate | EMOLLIENT | 1.2 |
| Hydrolyzed Jojoba Esters; | EMOLLIENT | 0.5 |
| Jojoba Esters; Water | | |
| Hydroxyacetophenone | SKIN CONDITIONER | 0.6 |
| Cocoglycerides | EMOLLIENT | 3.2 |
| Polyhydroxystearic Acid | DISPERSING AGENT- | 1.0 |
| | NONSURFACTANT | |

The SPF for each composition was measured using the IN VITRO SPF TEST METHOD. The ratio of SPF to total, combined weight percent of UV absorbers and UV blockers was calculated for each composition.

Table 6 shows the results, along with the amount of UV absorbers and UV blockers in each composition.

TABLE 6

| | | | Polyglyceryl | | | | | SPF/Wt |
|---|---|---|---|---|---|---|---|---|
| | | | benzotriazolyl | | | | | % UV |
| | | | t-butyl-hydroxyphenyl- | | | | | Absorbers |
| | TiO₂ | BEMT | propionate | EHT | MBBT | DHHB | | and |
| Composition | wt % | wt % | wt % | wt % | wt % | wt % | SPF | Blockers |
| 19 | 0.50 | 0.50 | 0 | 2.50 | 4.00 | 2.00 | 39.1 | 4.12 |
| 20 | 0.50 | 0.50 | 5.00 | 2.50 | 4.00 | 2.00 | 81.76 | 5.64 |
| 21 | 0.50 | 0.50 | 10.00 | 2.50 | 4.00 | 2.00 | 126.55 | 6.49 |
| 22 | 0.50 | 0.5 | 15.00 | 2.50 | 4.00 | 2.00 | 118.19 | 4.82 |
| 23 | 0.50 | 0.50 | 20.00 | 2.50 | 4.00 | 2.00 | 112.74 | 3.82 |
| 24 | 0 | 0 | 5.00 | 0 | 0 | 0 | 9.81 | 1.96 |
| 25 | 0 | 0 | 10.00 | 0 | 0 | 0 | 36.88 | 3.69 |
| 26 | 0 | 0 | 15.00 | 0 | 0 | 0 | 16.33 | 1.09 |

Compositions 19-23 containing a combination of UV absorbers and UV blockers according to the invention provided excellent in vitro SPF results of 39.1 to 126.55. The addition of polyglyceryl benzotriazolyl t-butylhydroxyphenylpropionate to Compositions 20-23 synergistically increased SPF versus Composition 19. The best SPF results were obtained using 10 or 15 weight percent of polyglyceryl benzotriazolyl t-butylhydroxyphenylpropionate in combination with BEMT, EHT, MBBT, DHHB and TiO2.

### Example 4

Compositions 27 to 31 according to the invention were prepared using the ingredients shown in Tables 7, 8, 9, and 10.

TABLE 7

| COMPOSITION 27 | | |
|---|---|---|
| | Percentage | |
| INCI Name | (% w/w) | Function |
| Water | 63.95 | Solvent |
| Disodium EDTA | 0.2 | Chelating Agent |
| Diethylamino Hydroxybenzoyl | 4 | UV Absorber |
| Hexyl Benzoate | | |
| Ethylhexyl Triazone | 4 | UV Absorber |
| Titanium Dioxide; Silica | 0.3 | UV Blocker |
| Hydrolyzed Jojoba Esters; | 0.3 | Film Former |
| Jojoba Esters; Water | | |
| C12-15 Alkyl Benzoate | 4 | Emollient |
| Diisopropyl Adipate | 3 | Emollient |
| Diisopropyl Sebacate | 1 | Emollient |
| Caprylyl Methicone | 3 | Emollient |
| Acrylates/C10-30 Alkyl | 0.15 | Viscosity |
| Acrylate Crosspolymer | | Increasing |
| | | Agent |
| Tocopheryl Acetate | 0.2 | Antioxidant |
| Potassium Cetyl Phosphate | 0.5 | Emulsifier |
| Panthenol | 0.1 | Skin Conditioner |
| Bis-Ethylhexyloxyphenol Methoxyphenyl | 0.5 | UV Absorber |
| Triazine | | |
| Triacontanyl PVP | 0.35 | Film Former |
| Glyceryl Stearate; PEG-100 Stearate | 1.5 | Emulsifier |
| Hydroxyethyl Acrylate/Sodium | 0.3 | Viscosity |
| Acryloyldimethyl Taurate Copolymer | | Increasing |
| | | Agent |
| Dimethicone; Dimethicone Crosspolymer | 2 | Skin Conditioner |
| Methylene bis-benzotriazolyl | 4 | UV Absorber |
| tetramethylbutylphenol | | |
| Hydroxyacetophenone | 0.7 | Antioxidant |
| Niacinamide | 2 | Skin Conditioner |
| Aluminum Starch Octenylsuccinate | 2.5 | Absorbent |
| Fragrance | 0.1 | Fragrance |
| Glycerin | 1 | Humectant |
| Sodium Hyaluronate | 0.05 | Humectant |
| Sodium Ascorbyl Phosphate | 0.2 | Antioxidant |
| Pentylene Glycol | 0.1 | Skin Conditioner |
| Sodium Hydroxide | 0.00 | pH Adjuster |

TABLE 8

| COMPOSITION 28 | | |
|---|---|---|
| | Percentage | |
| INCI Name | (% w/w) | Function |
| Water | 59.54 | Solvent |
| Pentylene Glycol | 1.00 | Skin |
| | | Conditioner |
| Iron Oxides; Sodium C14-16 | 0.43 | Colorant |
| Olefin Sulfonate | | |
| Titanium Dioxide; Sodium C14-16 | 2.97 | Colorant |
| Olefin Sulfonate | | |
| Iron Oxides; Sodium C14-16 | 0.08 | Colorant |
| Olefin Sulfonate | | |
| Sodium C14-16 Olefin Sulfonate; | 0.02 | Colorant |
| Iron Oxides | | |
| Diethylamino Hydroxybenzoyl | 4 | UV Absorber |
| Hexyl Benzoate | | |
| Ethylhexyl Triazone | 4 | UV Absorber |
| Bis-Ethylhexyloxyphenol | 0.5 | UV Absorber |
| Methoxyphenyl Triazine | | |
| Titanium Dioxide; Silica | 0.3 | UV Blocker |
| C12-15 Alkyl Benzoate | 4 | Emollient |
| Diisopropyl Adipate | 3 | Emollient |
| Diisopropyl Sebacate | 1 | Emollient |
| Caprylyl Methicone | 3 | Emollient |
| Hydrolyzed Jojoba Esters; | 0.3 | Film Former |
| Jojoba Esters; Water | | |
| Panthenol | 0.1 | Skin |
| | | Conditioner |
| Tocopheryl Acetate | 0.2 | Antioxidant |
| Dimethicone; Dimethicone | 2 | Skin |
| Crosspolymer | | Conditioner |
| Acrylates/C10-30 Alkyl | 0.15 | Viscosity |
| Acrylate Crosspolymer | | Increasing |
| | | Agent |
| Hydroxyethyl Acrylate/Sodium | 0.3 | Viscosity |
| Acryloyldimethyl Taurate Copolymer | | Increasing |
| | | Agent |
| Potassium Cetyl Phosphate | 0.5 | Emulsifier |
| Triacontanyl PVP | 0.35 | Film Former |
| Glyceryl Stearate; PEG-100 Stearate | 1.5 | Emulsifier |
| Disodium EDTA | 0.2 | Chelating |
| | | Agent |
| Hydroxyacetophenone | 0.7 | Antioxidant |
| Methylene bis-benzotriazolyl | 4 | UV Absorber |
| tetramethylbutylphenol | | |
| Niacinamide | 2.00 | Skin |
| | | Conditioner |
| Glycerin | 1.00 | Humectant |
| Sodium Hyaluronate | 0.05 | Humectant |
| Aluminum Starch Octenylsuccinate | 2.5 | Absorbent |
| Sodium Ascorbyl Phosphate | 0.2 | Antioxidant |
| Fragrance | 0.1 | Fragrance |
| Sodium Hydroxide | 0.01 | pH Adjuster |

TABLE 9

| COMPOSITION 29 | | |
|---|---|---|
| | Percentage | |
| INCI Name | (% w/w) | Function |
| Water | 59.54 | Solvent |
| Pentylene Glycol | 1.00 | Skin Conditioner |
| Iron Oxides; Sodium C14-16 | 1.25 | Colorant |
| Olefin Sulfonate | | |
| Sodium C14-16 Olefin Sulfonate; | 0.08 | Colorant |
| Iron Oxides | | |
| Iron Oxides; Sodium C14-16 | 0.20 | Colorant |
| Olefin Sulfonate | | |
| Titanium Dioxide; Sodium | 1.97 | Colorant |
| C14-16 Olefin Sulfonate | | |
| Diethylamino Hydroxybenzoyl | 4 | UV Absorber |
| Hexyl Benzoate | | |
| Ethylhexyl Triazone | 4 | UV Absorber |
| Bis-Ethylhexyloxyphenol | 0.5 | UV Absorber |
| Methoxyphenyl Triazine | | |
| Titanium Dioxide; Silica | 0.3 | UV Blocker |
| C12-15 Alkyl Benzoate | 4 | Emollient |
| Diisopropyl Adipate | 3 | Emollient |
| Diisopropyl Sebacate | 1 | Emollient |
| Caprylyl Methicone | 3 | Emollient |
| Panthenol | 0.1 | Skin Conditioner |
| Hydrolyzed Jojoba Esters; | 0.3 | Film Former |
| Jojoba Esters; Water | | |
| Tocopheryl Acetate | 0.2 | Antioxidant |
| Dimethicone; Dimethicone | 2 | Skin Conditioner |
| Crosspolymer | | |
| Acrylates/C10-30 Alkyl | 0.15 | Viscosity |
| Acrylate Crosspolymer | | Increasing Agent |
| Disodium EDTA | 0.2 | Chelating Agent |
| Hydroxyacetophenone | 0.7 | Antioxidant |
| Potassium Cetyl Phosphate | 0.5 | Emulsifier |
| Triacontanyl PVP | 0.35 | Film Former |
| Glyceryl Stearate; PEG-100 Stearate | 1.5 | Emulsifier |
| Niacinamide | 2 | Skin Conditioner |
| Hydroxyethyl Acrylate/Sodium | 0.3 | Viscosity |
| Acryloyldimethyl Taurate | | Increasing |
| Copolymer | | Agent |
| Glycerin | 1 | Humectant |
| Sodium Hyaluronate | 0.05 | Humectant |
| Methylene bis-benzotriazolyl | 4 | UV Absorber |
| tetramethylbutylphenol | | |
| Aluminum Starch Octenylsuccinate | 2.5 | Absorbent |
| Sodium Ascorbyl Phosphate | 0.2 | Antioxidant |
| Fragrance | 0.1 | Fragrance |
| Sodium Hydroxide | 0.01 | pH Adjuster |

TABLE 10

| | | COM - | COM- |
|---|---|---|---|
| | | POSITION | POSITION |
| | | 30 | 31 |
| | | Percentage | Percentage |
| INCI Name | Function | (% w/w) | (% w/w) |
| Triacontanyl PVP | FILM | 0.350000 | 0.350000 |
| | FORMING | | |
| | AGENT | | |
| Aluminum Starch | ABSORBENT | 2.000000 | 2.000000 |
| Octenylsuccinate; | | | |
| Aqua | | | |
| Acrylates/C10-30 | VISCOSITY | 0.200000 | 0.200000 |
| Alkyl Acrylate | INCREASING | | |
| Crosspolymer | | | |
| Disodium EDTA | CHELATING | 0.100000 | 0.100000 |
| | AGENT | | |
| Methylene bis- | UV | 5.000000 | 5.000000 |
| benzotriazolyl | ABSORBER | | |
| tetramethyl- | | | |
| butylphenol | | | |
| (nano); Aqua; Decyl | | | |
| Glucoside: Propylene | | | |
| Glycol; Xanthan Gum | | | |
| Sodium Hydroxide | PH ADJUSTER | 0.050000 | 0.050000 |
| Potassium Cetyl | EMULSIFIER | 1.200000 | 1.200000 |
| Phosphate | | | |
| Silica | ABSORBENT | 1.000000 | 1.000000 |
| Ethylhexyl Triazone | UV | 4.000000 | 4.000000 |
| | ABSORBER | | |
| Aqua | SOLVENT | 39.310000 | 39.310000 |
| Caprylyl Methicone | EMOLLIENT | 2.000000 | 2.000000 |
| Titanium Dioxide | UV Blocker | 0.300000 | 0.300000 |
| (nano); Silica | | | |
| Sodium Polyacrylate | VISCOSITY | 0.200000 | 0.200000 |
| | INCREASING | | |
| Cetyl Alcohol; | EMULSIFIER | 2.000000 | 2.000000 |
| Glyceryl | | | |
| Stearate; PEG-75 | | | |
| Stearate; Ceteth-20; | | | |
| Steareth-20 | | | |
| Diethylamino | UV | 4.500000 | 4.500000 |
| Hydroxybenzoyl | ABSORBER | | |
| Hexyl | | | |
| Benzoate | | | |
| Diisopropyl Sebacate | EMOLLIENT | 2.100000 | 2.100000 |
| Diisopropyl Adipate | EMOLLIENT | 2.600000 | 2.600000 |
| Glycerin | HUMECTANT | 2.000000 | 2.000000 |
| Bis- | UV | 1.800000 | 1.800000 |
| Ethylhexyloxyphenol | ABSORBER | | |
| Methoxyphenyl | | | |
| Triazine | | | |
| CI 77492; Sodium | COLORANT | 0.750000 | 1.842000 |
| C14-16 Olefin | | | |
| Sulfonate | | | |
| CI 77891; Sodium | COLORANT | 5.160000 | 3.759300 |
| C14-16 Olefin | | | |
| Sulfonate | | | |
| Feverfew Extract | SKIN | 1.000000 | 1.000000 |
| (Chrysanthemum | CONDITIONER | | |
| Parthenium | | | |
| Flower/Leaf/Stem | | | |
| Juice; Limonene; | | | |
| Farnesol) | | | |
| CI 77499; Sodium | COLORANT | 0.030000 | 0.112800 |
| C14-16 Olefin | | | |
| Sulfonate | | | |
| CI 77491; Sodium | COLORANT | 0.150000 | 0.375900 |
| C14-16 Olefin | | | |
| Sulfonate | | | |
| C12-15 Alkyl | SOLUBILIZER | 9.800000 | 9.800000 |
| Benzoate; Benzyl | | | |
| Alcohol; Benzyl | | | |
| Benzoate | | | |
| Hydrolyzed Jojoba | EMOLLIENT | 0.500000 | 0.500000 |
| Esters; Jojoba Esters; | | | |
| Aqua | | | |
| Dimethicone; | SKIN | 1.500000 | 1.500000 |
| Dimethicone | CONDITIONER | | |
| Crosspolymer | | | |
| ACETYL | SKIN | 4.000000 | 4.000000 |
| GLUCOSAMINE | CONDITIONER | | |
| Stearyl Alcohol; | VISCOSITY | 1.500000 | 1.500000 |
| Arachidyl Alcohol; | INCREASING | | |
| Cetyl Alcohol | | | |
| Niacinamide | SKIN | 4.000000 | 4.000000 |
| | CONDITIONER | | |
| Parfum | FRAGRANCE | 0.300000 | 0.300000 |
| Hydroxy- | SKIN | 0.600000 | 0.600000 |
| acetophenone | CONDITIONER | | |
| | Total | 100.000000 | 100.000000 |

The Compositions 27 to 31 were found to provide excellent moisturization to skin when topically applied. In addition, advantageously, the UV absorbers and UV blockers in the compositions remained substantially on top of the skin, i.e., did not permeate the skin, and therefore provided excellent UV protection. On the other hand, the cosmetically acceptable active agents such as niacinamide permeated the skin. In this manner, the efficacy of the UV absorbers and UV blockers, on the one hand, and the efficacy of the cosmetically acceptable active agents, on the other, were both preserved.

We claim:

1. A sunscreen composition comprising: (a) diethylamino hydroxybenzoyl hexyl benzoate; (b) bis-ethylhexyloxyphenol methoxyphenyl triazine; (c) ethylhexyl triazone; and (d) methylene bis-benzotriazolyl tetramethylbutylphenol; wherein the sunscreen composition is substantially free of UV absorbers having a molecular mass of 380 Daltons or less.

2. The composition of claim 1 further comprising a UV blocker selected from the group consisting of titanium dioxide and zinc oxide.

3. The composition of claim 2, wherein the UV blocker is titanium dioxide.

4. The composition of claim 1 further comprising tris-biphenyl triazine.

5. The composition of claim 1 further comprising a polymeric UV absorber.

6. The composition of claim 5, wherein the polymeric UV absorber is a linear UV-absorbing polyether of the following formula: 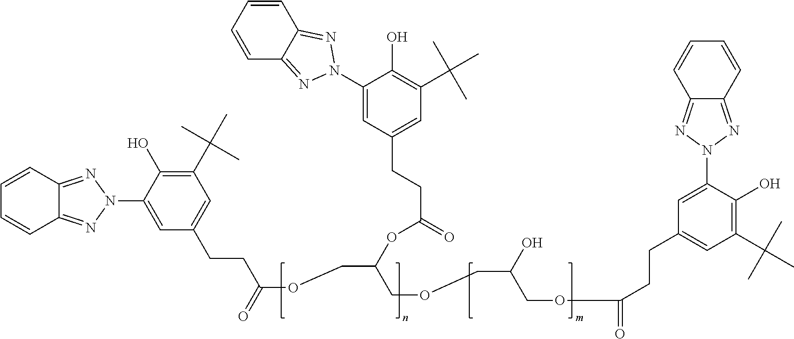
wherein n and m, independently from each other, are each a number from 0 to 20;
and at least one of m and n is >1.

7. The composition of claim 1 having an SPF of at least about 30 as measured by the IN VITRO SPF TEST METHOD, wherein the ratio of the SPF of the composition to the total, combined weight percent of UV absorbers and UV blockers in the composition is at least about 4.1.

8. The composition of claim 1 further comprising a cosmetically acceptable topical carrier.

9. The composition of claim 8 further comprising a cosmetically acceptable active agent selected from the group consisting of from niacinamide, tocopheryl acetate, ascorbic acid, acetyl glucosamine, trehalose, feverfew extract, glycerin, sodium hyaluronate and capryloyl glycine (and) sarcosine (and) Cinnamomum seylanicum bark extract, and mixtures thereof.

10. The composition of claim 9, wherein the cosmetically acceptable active agent is selected from the group consisting of niacinamide, glycerin, and sodium hyaluronate.

11. The composition of claim 9, wherein the cosmetically acceptable active agent is selected from the group consisting of acetyl glucosamine, niacinamide, and mixtures thereof.

12. A sunscreen composition comprising:
(a) about 2 to about 6 weight percent of diethylamino hydroxybenzoyl hexyl benzoate;
(b) 3 weight percent or less of tris-biphenyl triazine;
(c) about 0.5 to about 3.5 weight percent of bis-ethylhexyloxyphenol methoxyphenyl triazine;
(d) about 1.64 to about 4.55 weight percent of ethylhexyl triazone;
(e) about 2 to about 6 weight percent of methylene bis-benzotriazolyl tetramethylbutylphenol; and
(f) 2 weight percent or less of titanium dioxide;
wherein the sunscreen composition is substantially free of UV absorbers having a molecular mass of 380 Daltons or less.

13. The composition of claim 12 further comprising about 1 to about 5 weight percent of niacinamide, about 0.5 to about 2 weight percent glycerin, and about 0.01 to about 0.1 weight percent of sodium hyaluronate.

14. The composition of claim 12 further comprising about 2 to about 6 weight percent of acetyl glucosamine and about 1 to about 5 weight percent of niacinamide.

15. A sunscreen composition comprising UV absorbers, UV blockers and a cosmetically acceptable topical carrier, wherein the UV absorbers and UV blockers consist of:
(a) about 2 to about 6 weight percent of diethylamino hydroxybenzoyl hexyl benzoate;
(b) 3 weight percent or less of tris-biphenyl triazine;
(c) about 0.5 to about 3.5 weight percent of bis-ethylhexyloxyphenol methoxyphenyl triazine;
(d) about 1.64 to about 4.55 weight percent of ethylhexyl triazone;
(e) about 2 to about 6 weight percent of methylene bis-benzotriazolyl tetramethylbutylphenol; and
(f) 2 weight percent or less of titanium dioxide.

16. The composition of claim 15 further comprising about 1 to about 5 weight percent of niacinamide, about 0.5 to about 2 weight percent glycerin, and about 0.01 to about 0.1 weight percent of sodium hyaluronate.

17. The composition of claim 15 further comprising about 2 to about 6 weight percent of acetyl glucosamine and about 1 to about 5 weight percent of niacinamide.
